(19) United States
(12) Patent Application Publication
(10) Pub. No.: US 2023/0109605 A1
(43) Pub. Date: Apr. 6, 2023

(54) SYSTEMS AND METHODS FOR IMPROVED RETENTION OF STENTS ON A DELIVERY SYSTEM

(71) Applicant: Cook Medical Technologies LLC, Bloomington, IN (US)

(72) Inventors: David C. Majercak, Bloomington, IN (US); Matt Wildridge, Stinesville, IN (US); Ellie Glover, Fairfield, OH (US); Mark Svendsen, Bloomington, IN (US)

(73) Assignee: Cook Medical Technologies LLC, Bloomington, IN (US)

(21) Appl. No.: 17/903,708

(22) Filed: Sep. 6, 2022

Related U.S. Application Data
(62) Division of application No. 16/939,549, filed on Jul. 27, 2020, now abandoned.

(51) Int. Cl. A61F 2/958

(52) U.S. Cl. CPC A61F 2/958; A61F 2002/9505

(57) ABSTRACT

The present embodiments provide systems and methods for improved retention of stents on a delivery system. In one embodiment, a delivery system comprises a catheter and a balloon. A proximal end of the balloon is secured to an exterior surface of the catheter at a first location, and a distal end of the balloon is secured to the exterior surface of the catheter at a second location. A plurality of bands are disposed in a circumferential space situated between the exterior surface of the catheter and an interior surface of the balloon. The plurality of bands may comprise at least four bands that are discretely spaced-apart in an axial direction from one another along a length of the catheter. At least one stent is secured to the exterior surface of the balloon in a compressed delivery state.

### CROSS-REFERENCE To RELATED APPLICATIONS

The present patent application is a divisional of U.S. patent application Ser. No. 16/939,549, filed Jul. 27, 2020, which is incorporated by reference in its entirety.

### BACKGROUND

The present embodiments relate generally to apparatus and methods for treating medical conditions, and more specifically, to stents or stent-grafts for use in body vessels to treat those medical conditions.

Stents may be inserted into an anatomical vessel or duct for various purposes. Stents may maintain or restore patency in a formerly blocked or constricted passageway, for example, following a balloon angioplasty procedure. Other stents may be used for different procedures, for example, stents placed in or about a graft have been used to hold the graft in an open configuration to treat an aneurysm. Additionally, stents coupled to one or both ends of a graft may extend proximally or distally away from the graft to engage a healthy portion of a vessel wall away from a diseased portion of an aneurysm to provide endovascular graft fixation.

Stents may be either self-expanding or balloon-expandable, or they can have characteristics of both types of stents. Self-expanding stents may be delivered to a target site in a compressed configuration and subsequently expanded by removing a delivery sheath, removing trigger wires and/or releasing diameter reducing ties. With self-expanding stents, the stents expand primarily based on their own expansive force without the need for further mechanical expansion. In a stent made of a shape-memory alloy such as nitinol, the shape-memory alloy may be employed to cause the stent to return to a predetermined configuration upon removal of the sheath or other device maintaining the stent in its predeployment configuration. In contrast, with balloon-expandable stents, the expansion force of an inflatable balloon causes the stent to expand.

In prior balloon-expandable systems, securement of a stent to a balloon catheter has experienced challenges. Often, a securement technique known as “pillowing” is employed, which uses a slightly larger inner diameter tube with high pressure and increased heat, intended to drive the balloon into the openings in the stent. While this technique may improve stent security, it increases the delivery profile.

Additionally, there may be accuracy challenges when deploying or flaring a stent using a balloon, due to movement of the stent along the length of the balloon. Further, in balloon-expandable systems, it may be challenging to secure relatively short stents to a balloon catheter.

### SUMMARY

The present embodiments provide systems and methods for improved retention of stents on a delivery system. In one embodiment, a delivery system for use in a medical procedure comprises a catheter having proximal and distal regions and an exterior surface, and further comprises a balloon having proximal and distal ends and having interior and exterior surfaces. The proximal end of the balloon is secured to the exterior surface of the catheter at a first location, and the distal end of the balloon is secured to the exterior surface of the catheter at a second location, wherein the second location is distal to the first location.

The system further comprises a plurality of bands, where each of the plurality of bands are disposed in a circumferential space situated between the exterior surface of the catheter and the interior surface of the balloon. In this example, the plurality of bands comprise at least four bands that are discretely spaced-apart in an axial direction from one another along a length of the catheter. The system further comprises at least one stent having a compressed delivery state and an expanded deployed state, where the at least one stent is secured to the exterior surface of the balloon in the compressed delivery state.

In one example, each of the plurality of bands may be secured directly to the exterior surface of the catheter. In one embodiment, the plurality of bands are formed from silicone.

In one embodiment, the at least one stent may be positioned at a location radially overlapping a first band of the plurality of bands in the compressed delivery state. In another example, the at least one stent may be positioned axially between first and second bands of the plurality of bands in the compressed delivery state.

In one example, the system comprises a covered stent having a covering including proximal and distal ends, wherein the at least one stent is disposed along a length of the covering. In this example, multiple stents may be discretely spaced-apart in an axial direction from one another between the proximal and distal ends of the covering. The covering may comprise expanded PTFE (ePTFE), and the multiple stents may be encapsulated in the ePTFE. The at least one stent may comprise a z-shape having a plurality of proximal and distal apices separated by a plurality of angled strut segments.

The present embodiments further comprise a method for securing at least one stent to a catheter. The method comprises providing a catheter having a balloon secured to an exterior surface of the catheter at first and second locations, wherein a plurality of bands are disposed in a circumferential space situated between the exterior surface of the catheter and the interior surface of the balloon. The plurality of bands comprise at least four bands that are discretely spaced-apart in an axial direction from one another along a length of the catheter. The method comprises crimping at least one stent to the exterior surface of the balloon, wherein upon crimping the at least stent engages at least one of the plurality of bands to enhance securement of the at least one stent relative to the catheter in the compressed delivery state.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side view of a first embodiment of a deployment system including a plurality of bands disposed beneath a balloon, where the balloon is depicted as clear to show components therein.

FIG. 2 is a side view of the system of FIG. 1 with selected interior components depicted in dashed lines.

FIG. 3 is an isolated perspective view of an exemplary band of the delivery system of FIGS. 1-2.

FIG. 4 is a side view of an example of a covered stent suitable for use with the delivery system of FIGS. 1-3.

FIGS. 5A-5B are schematic side views illustrating at least one stent radially overlapping a respective band of the delivery system of FIGS. 1-2 in compressed and deployed states, respectively.

FIGS. 6A-6B are schematic side views illustrating at least one stent positioned axially between bands of the delivery system of FIGS. 1-2 in compressed and deployed states, respectively.

### DETAILED DESCRIPTION OF THE PREFERRED EMBODIMENTS

In the present application, the term “proximal end” is used when referring to that end of a medical device closest to an operator during a medical procedure, while the term “distal end” is used when referring to that end of a medical device furthest from an operator during a medical procedure.

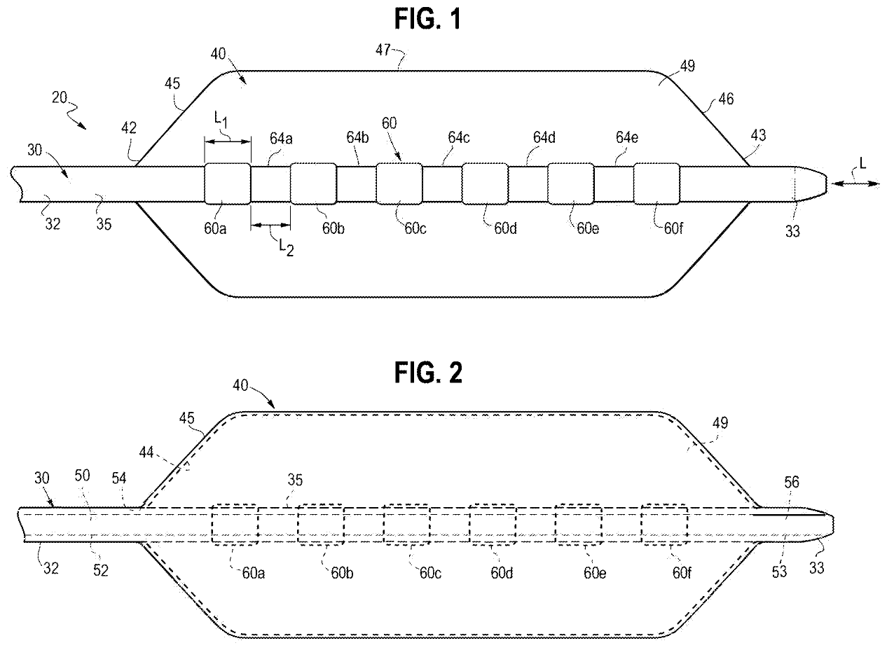

Referring now to FIGS. 1-2, a first embodiment of a delivery system 20 for use in a medical procedure is shown and described. The delivery system 20 comprises a catheter 30 having proximal and distal regions 32 and 33, and an exterior surface 35. The system further comprises a balloon 40 having proximal and distal ends 42 and 43, and further having an interior surface 44 and an exterior surface 45, as depicted in FIGS. 1-2. In one exemplary technique, the proximal end 42 of the balloon 40 is secured to the exterior surface 35 of the catheter 30 at a first location, and the distal end 43 of the balloon 40 is secured to the exterior surface 35 of the catheter 30 at a second location, where the second location is distal to the first location, as shown in FIGS. 1-2. The balloon 40 may be affixed to the exterior surface 35 of the catheter 30 using an adhesive, such a biocompatible glue, or alternatively, using a heat-bond, heat-shrink tubing, one or more tie-down bands, or the like.

In one example, the catheter 30 may be formed from one or more semi-rigid polymers and the balloon 40 may be manufactured from any suitable balloon material used during an interventional procedure, such as PEBAX, nylon, Hytrel, Arnitel, or other polymers. The balloon 40 comprises uninflated and inflated states. As explained further below, a stent or stent-graft may be placed over the balloon 40 and aligned with the balloon when the balloon is in the uninflated state. Subsequently, an inflation fluid may be provided into the inner confines of the balloon 40 to expand the balloon to the inflated state, as shown in FIG. 2.

In one embodiment, an inner shaft member 50 having proximal and distal ends 52 and 53 extends within a lumen of the catheter 30. The delivery system 20 may be configured such that an outer diameter of inner shaft member 50 is smaller than an inner diameter of the catheter 30, thereby creating a cavity forming an inflation lumen 54, as depicted in FIG. 2, which may be placed in fluid communication with an interior surface of the balloon 40. In the embodiment depicted, where the distal region 33 of the catheter 30 terminates distal to the balloon 40, one or more apertures in the catheter 30 may permit fluid communication into the balloon 40. Alternatively, the distal region 33 of the catheter 30 may terminate within the confines of the balloon 40, and the inner shaft member 50 may extend by itself distally beyond the balloon 40 and may be secured directly to the distal end 43 of the balloon 40, such that inflation fluid delivered via inflation lumen 54 is channeled into the confines of the balloon 40 upon exiting the distal region 33 of the catheter 30.

The inner shaft member 50 may comprise a hollow tubular member having an inner lumen 56 formed therein, as shown in FIG. 2. The inner lumen 56 may span between the proximal and distal ends 52 and 53 of the inner shaft member 50 and may be configured to receive a wire guide and/or other medical components. In one exemplary method of use, a wire guide may be delivered within a vessel to a site of a vascular condition, and the delivery system 20 may be delivered over the wire guide by placing the distal end 53 of the inner shaft member 50 over the wire guide and advancing the catheter 30 distally while the balloon 40 is in a deflated state.

The balloon 40 may comprise any number of configurations. In the embodiments depicted herein, the balloon 40 comprises a proximal taper 45, a distal taper 46, and a central region 47 formed therebetween, as shown in FIG. 1. Alternatively, the balloon 40 may have proximal and distal regions and comprise other shapes, for example, a substantially circular shape, oval shape, tapered or stepped shape, or the like. In the exemplary embodiment shown herein, the central region 47 may be sized and configured to perform angioplasty or another desired procedure upon an inner wall of a vessel, and preferably has an axial length sufficient to accommodate placement of a plurality of bands 60 beneath the balloon 40, as explained further below.

The delivery system 20 further comprises the plurality of bands 60. In the non-liming embodiment of FIGS. 1-2, six bands 60a-60f are depicted. It will be appreciated that this is for illustrative purposes only, and that greater or fewer than six bands may be provided. However, the inventors have experimentally determined that it may be desirable to include at least four different bands, to achieve significant advantages as explained further below.

Each of the plurality of bands 60 are disposed in a circumferential space 49 situated between the exterior surface 35 of the catheter 30 and the interior surface 44 of the balloon 40, as depicted in FIGS. 1-2. In a presently preferred embodiment, the plurality of bands 60 are discretely spaced-apart in an axial direction from one another along a longitudinal axis L of the catheter 30, as best seen in FIG. 1. In the example where six bands 60a-60f are provided, then there are five spaces 64a-64e provided between the bands, as shown in FIG. 1.

In the example shown, the plurality of bands 60a-60f each comprise a generally identical shape with an axial length L₁. However, it should be understood that the plurality of bands 60a-60f may comprises different shapes relative to one another, and particularly different axial lengths, without departing from the present embodiments. Similarly, it should be understood that while the spaces 64a-64e are generally depicted as having similar axial lengths L₂, the spaces 64a-64e may comprise different axial lengths relative to one another, or relative to the axial lengths L₁ of the bands, without departing from the present embodiments.

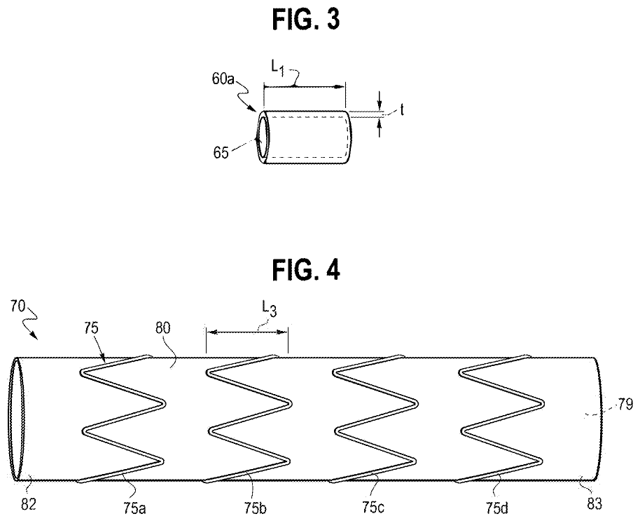

In one embodiment, each of the plurality of bands 60 are secured directly to the exterior surface 35 of the catheter 30. The plurality of bands 60 may have a degree of elasticity that enables an interior region 65 of the bands 60, depicted in FIG. 3, to assume a slightly larger diameter than the exterior surface 35 of the catheter 30 for sliding of the bands 60 over the catheter, yet when relaxed the bands will assume a smaller diameter that frictionally engages the exterior surface 35 of the catheter 30. Such frictional engagement may be sufficient to secure the bands 60 relative to the catheter 30, or alternatively or in addition, the bands 60 may be secured using other mechanical, adhesive or thermal techniques. In some embodiments, the bands 60 may originate from a non-tubular piece that is then wound around the exterior surface 35 of the catheter and affixed in place in a circular form in the manner shown in FIGS. 1-3. In one embodiment, the plurality of bands are formed from silicone.

The delivery system 20 further comprises at least one stent having a compressed delivery state and an expanded deployed state, wherein the at least one stent is secured to the exterior surface 35 of the balloon 30 in the compressed delivery state. The at least one stent may be a stand-alone stent, or may be part of a covered stent or a stent-graft.

Referring to FIG. 4, an exemplary covered stent 70, which is suitable for use with the delivery system 20, is shown in an isolated manner prior to being secured to the balloon catheter of FIGS. 1-2. It will be appreciated that while a covered stent 70 is generally shown, an uncovered stent, or partially covered and uncovered stent, may alternatively be used. In the examples of a covered stent 70 shown, the covered stent 70 comprises at least one stent 75 and a covering 80. As used in the present application, the term “covering” generally refers to the provision of one or more layers of material that are separate from the stent itself. The covering 80 need not be disposed external to the stent 75, for example, the covering may be generally disposed internal to the stent 75, or the stent 75 may be at least partially embedded in the covering 80.

The covered stent 70 may be used in a wide range of procedures, for example, to treat an aneurysm, stenosis or other condition. The stent 75 generally provides radial force needed to expand the covered stent 70 into engagement at a target site, while the covering 80 may provide a barrier having a selected porosity and may be suitable for delivering one or more therapeutic agents. A lumen 79 may be formed internal to the covering 80 and may be suitable for carrying fluid though the covered stent 70.

The at least one stent 75 may be made from numerous metals and alloys. In one example, the stent 75 comprises a shape-memory material such as a nickel-titanium alloy (“nitinol”). Moreover, the structure of the stent 75 may be formed in a variety of ways to provide a suitable intraluminal support structure. For example, one or more stents 75 may be made from a woven wire structure, a laser-cut cannula, individual interconnected rings, or another pattern or design.

In one example, as depicted in FIG. 4, the at least one stent 75 may be configured in the form of one or more “Z-stents” or Gianturco stents, each of which may comprise a series of substantially straight segments interconnected by a series of bent segments. The bent segments may comprise acute bends or apices. The Gianturco stents are arranged in a zigzag configuration in which the straight segments are set at angles relative to each other and are connected by the bent segments. In the example depicted herein, four different Z-stents 75a-75d are depicted as being discretely spaced-apart in an axial direction from one another between proximal and distal ends 82 and 83 of the covering 80, as shown in FIG. 4.

The covering 80 may comprise a polymeric sheet having any suitable porosity. The porosity may be substantially porous or substantially non-porous and may be selected depending on the application. In one example, a porous polymeric sheet may comprise the polyurethane Thoralon®. In addition to, or in lieu of, a porous polyurethane, the covering 80 may comprise any biocompatible polymeric material including non-porous polyurethanes, PTFE, expanded PTFE (ePTFE), polyethylene tetraphthalate (PET), aliphatic polyoxaesters, polylactides, polycaprolactones, and hydrogels. The coating also may comprise a graft material, such as Dacron®, which may optionally be heat treated and/or partially melted.

The one or more stents 75 have a compressed, reduced diameter delivery state in which the covered stent 70 may be advanced to a target location within a vessel, duct or other anatomical site. The one or more stents 75 further have an expanded state, as shown in FIG. 4, in which they may be configured to apply a radially outward force upon the vessel, duct or other target location, e.g., to maintain patency within a passageway. In the expanded state, fluid flow is allowed through the lumen 79 of the coated stent 70.

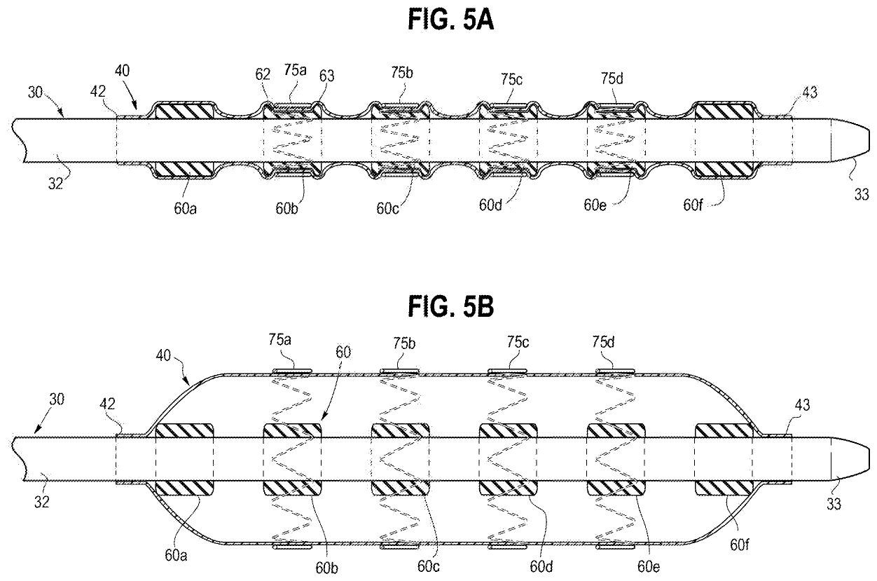

Referring now to FIGS. 5A-5B, a first embodiment depicting engagement of the at least one stent with the delivery system 20 of FIGS. 1-2 is shown and described. In this embodiment, at least one stent 75 is positioned at a location radially overlapping a first band of the plurality of bands 60 in the compressed delivery state. For example, the stent 75 is positioned to radially overlap with the second band 60b in the compressed delivery state of the stent 75, as shown in FIG. 5A.

In the example where multiple stents 75 are provided, e.g., such as the stents 75a-75d as part of the covered stent 70 of FIG. 4, then multiple stents may radially overlap with respective bands. As depicted in FIG. 5A, each of the stents 75a-75d radially overlaps with a respective band 60b-60e of the plurality of bands in the compressed delivery state.

In an exemplary method of FIG. 5A, the plurality of stents 75a-75d may be crimped towards the exterior surface of the balloon 40, where upon crimping the plurality of stents 75a-75d engage their respective bands 60b-60e to enhance securement of stents relative to the catheter 30 in the compressed delivery state.

Notably, if the plurality of bands 60a-60f are formed from a material such as silicone, then such material provides a barrier that “grips” the respective stents 75a-75d on their sides. More particularly, as depicted in FIG. 5A, a proximal region 62 of the band 60b may effectively grip a proximal region of the stent 75a, while a distal region 63 of the band 60b may effectively grip a distal region of the stent 75a. When crimped, the band 60b may further wrap around stent strut segments that are centrally located, i.e., between the proximal and distal apices of the stent 75a, thus effectively securing most or all of the stent 75a to the band 60b.

In one embodiment, an axial length L₃ of each stent 75a-75d, as measured in the manner shown in FIG. 4, may be substantially identical to the axial lengths L₁ of its respective band 60, which may maximize the surface area to which the stent may engage the band. Alternatively, the axial length L₃ of each stent 75a-75d may be less than the axial length L₁ of its respective band to facilitate “gripping” of the proximal and distal ends 62 and 63 of the bands 60 upon crimping, as depicted in FIG. 5A.

Advantageously, in this manner, the at least one stent 75 may be held securely adjacent to the catheter 30, on the exterior of the balloon 40, in the compressed delivery state of the stent 75. Moreover, if the at least one stent 75 is part of the covered stent 70 of FIG. 4 (it is noted that the covering 80 is omitted in FIG. 5A-5B for illustrative purposes), then the entire covered stent 70 may be effectively held adjacent to the catheter 30 on the exterior of the balloon 40.

Referring to FIG. 5B, when the balloon 40 is inflated, e.g., using the techniques described above, then such radially outward force may be sufficient to transition the at least one stent 75 from the compressed delivery state to the expanded deployed state. At this time, the at least one stent 75 becomes disengaged from its respective band 60, and may perform its desired function within a vessel or duct.

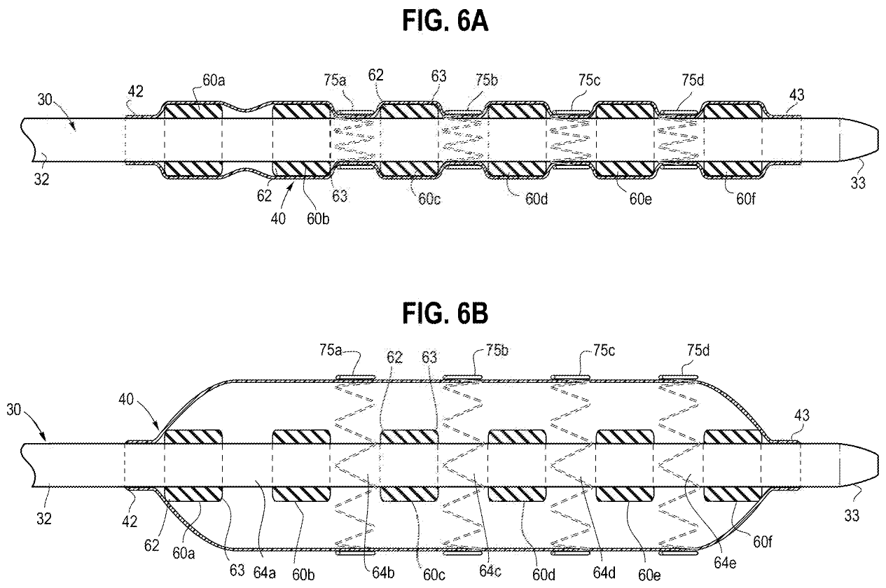

Referring now to FIGS. 6A-6B, an alternative embodiment depicting engagement of the at least one stent with the delivery system 20 of FIGS. 1-2 is shown and described. In this alternative embodiment, at least one stent 75 is positioned axially between first and second bands of the plurality of bands in the compressed delivery state. For example, the stent 75a is positioned axially between the second band 60b and the third band 60c in the compressed delivery state of the stent 75a, as shown in FIG. 6A.

In the example where multiple stents 75 are provided, e.g., such as the stents 75a-75d as part of the covered stent 70 of FIG. 4, then a first stent 75a may be positioned axially between second and third bands 60b and 60c, while a second stent 75b may be positioned axially between the third band 60c and a fourth band 60d in the compressed delivery state, a third stent 75c may be positioned axially between the fourth band 60d and a fifth band 60e in the compressed delivery state, and a fourth stent 75d may be positioned axially between the fifth band 60e and a sixth band 60f, as depicted in FIG. 6A. If additional stents 75 are provided, e.g., as part of the covered stent 70, then they may be positioned axially between additional bands in a similar manner.

In an exemplary method of FIG. 6A, the plurality of stents 75a-75d may be crimped towards the exterior surface of the balloon 40, where upon crimping the plurality of bands 60 act together to provide a “gripping” action that secures the plurality of stents 75a-75d relative to the catheter 30 in the compressed delivery state. More specifically, the distal region 63 of the band 60b works in conjunction with the proximal region 62 of the adjacent band 60c to jointly “grip” the stent 75a primarily in-between the adjacent bands 60b and 60c. Similar functionality occurs between the other stents 75a-75d and the respective bands for which they are disposed in-between.

In one embodiment, an axial length L₃ of each stent 75a-75d, as measured in the manner shown in FIG. 4, may be substantially identical to the axial lengths L₂ of the spaces 64a-64e in-between adjacent bands 60, which may promote a sandwich-like effect to enhance securement of a given stent 75 within its respective space 64. Alternatively, the axial length L₃ of each stent 75a-75d may be a small percentage greater or less than the axial lengths L₂ of the spaces 64a-64e, and upon crimping the same securement effect may be achieved.

Advantageously, in this manner, the at least one stent 75 may be held securely adjacent to the catheter 30, on the exterior of the balloon 40, in the compressed delivery state of the stent 75. Moreover, if the at least one stent 75 is part of the covered stent 70 of FIG. 4 (it is noted that the covering 80 is omitted in FIG. 6A-6B for illustrative purposes), then the entire covered stent 70 may be effectively held adjacent to the catheter 30 on the exterior of the balloon 40.

Referring to FIG. 6B, when the balloon 40 is inflated, e.g., using the techniques described above, then such radially outward force may be sufficient to transition the at least one stent 75 from the compressed delivery state to the expanded deployed state. At this time, the at least one stent 75 becomes disengaged from the space in-between its respective bands 60, and may perform its desired function within a vessel or duct.

Advantageously, both the embodiments of FIGS. 5A-5B and FIGS. 6A-6B encompass significant advantages relative to known securement techniques. First, a significantly improved retention force is provided. During experimental testing, stent retention of samples improved from about 5 N (prior to use of the bands 60) to approximately 20 N (with use of the bands as shown in the embodiments of FIGS. 5A-5B and FIGS. 6A-6B). In other words, the retention force improved by a factor of four by provision of the bands 60 during experimental testing. This improved level is generally enough such that a user cannot manually pull the stents 75 off the balloon 40.

Notably, such improved securement techniques may facilitate the delivery of relatively short stents, which may not have been able to be delivered previously due to an inability to be secured effectively to an exterior of a balloon.

As another advantage, such securement techniques may allow for a reduced delivery profile by obviating the need for pillowing, e.g., using a slightly larger inner diameter tube with high pressure and increased heat, which is used to drive the balloon into the openings in the stent. While this technique may improve stent security, it increases the delivery profile.

The present embodiments have a minimal effect, if any, on the overall profile of the system 20, and are compatible for use in a 6 French sheath system for stent having diameters, without limitation, in the range of about 5 mm to about 8 mm.

As yet another advantage, the present embodiments may facilitate deployment in a precise manner with one balloon, without movement of the stent along the length of the balloon.

It will be appreciated that although FIGS. 5A-5B and 6A-6B respectively show embodiments with either overlap of each stent 75 with a respective band or placement of each stent 75 in-between bands 60, in alternative embodiments selected stents 75a-75d may overlap with a given band 60 while other stents may be positioned in-between bands. In other words, a hybrid approach to FIGS. 5A-5B and 6A-6B may be used without departing from the present embodiments. Further, it will be appreciated that in alternative embodiments, selected stents 75a-75d may individually have a partial overlap with a given band 60 while partially being axially beyond the same band 60, such that a stent is partly on and partly off a particular band, without departing from the scope of the present embodiments.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

We claim:

1. A delivery system for use in a medical procedure, comprising:
a catheter having proximal and distal regions, and an exterior surface;
a balloon having proximal and distal ends, and further having interior and exterior surfaces,
wherein the proximal end of the balloon is secured to the exterior surface of the catheter at a first location, and wherein the distal end of the balloon is secured to the exterior surface of the catheter at a second location, wherein the second location is distal to the first location;
a plurality of bands, wherein each of the plurality of bands are disposed in a circumferential space situated between the exterior surface of the catheter and the interior surface of the balloon,
wherein the plurality of bands comprise at least four bands that are discretely spaced-apart in an axial direction from one another along a length of the catheter; and
at least one stent having a compressed delivery state and an expanded deployed state, wherein the at least one stent is secured to the exterior surface of the balloon in the compressed delivery state.

2. The delivery system of claim 1, wherein the at least one stent is positioned axially between first and second bands of the plurality of bands in the compressed delivery state.

3. The delivery system of claim 1, further comprising a covered stent having a covering including proximal and distal ends, wherein the at least one stent is disposed along a length of the covering.

4. The delivery system of claim 3, wherein multiple stents are discretely spaced-apart in an axial direction from one another between the proximal and distal ends of the covering.

5. The delivery system of claim 4, wherein a first stent of the multiple stents is positioned axially between first and second bands of the plurality of bands, and wherein a second stent of the multiple stents is positioned axially between the second band and a third band of the plurality of bands in the compressed delivery state.

6. A delivery system for use in a medical procedure, comprising:
a catheter having proximal and distal regions, and an exterior surface;
a balloon having proximal and distal ends, and further having interior and exterior surfaces,
wherein the proximal end of the balloon is secured to the exterior surface of the catheter at a first location, and wherein the distal end of the balloon is secured to the exterior surface of the catheter at a second location, wherein the second location is distal to the first location;
a plurality of bands, wherein each of the plurality of bands are disposed in a circumferential space situated between the exterior surface of the catheter and the interior surface of the balloon;
a covered stent having a covering including proximal and distal ends, wherein multiple stents are discretely spaced-apart in an axial direction from one another between the proximal and distal ends of the covering,
wherein the multiple stents each have a compressed delivery state and an expanded deployed state, and wherein the multiple stents are secured to the exterior surface of the balloon in the compressed delivery state.

7. The delivery system of claim 6, wherein a first stent of the multiple stents is positioned axially between first and second bands of the plurality of bands, and wherein a second stent of the multiple stents is positioned axially between the second band and a third band of the plurality of bands in the compressed delivery state.

8. A method for securing at least one stent to a catheter, comprising:
providing a catheter having a balloon secured to an exterior surface of the catheter at first and second locations, and wherein a plurality of bands are disposed in a circumferential space situated between the exterior surface of the catheter and the interior surface of the balloon,
wherein the plurality of bands comprise at least four bands that are discretely spaced-apart in an axial direction from one another along a length of the catheter; and
crimping at least one stent to the exterior surface of the balloon, wherein upon crimping the at least stent engages at least one of the plurality of bands to enhance securement of the at least one stent relative to the catheter in the compressed delivery state.
